(19) United States
(12) Patent Application Publication
(10) Pub. No.: US 2009/0062395 A1
(43) Pub. Date: Mar. 5, 2009

(54) RENIN INHIBITORS FOR THE TREATMENT OF HYPERTENSION

(76) Inventor: Andrew Satlin, Short Hills, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/293,107

(22) PCT Filed: Mar. 30, 2007
(86) PCT No.: PCT/US07/65564

§ 371 Date: Sep. 16, 2008

Related U.S. Application Data
(60) Provisional application No. 60/788,842, filed on Apr. 3, 2006.

(51) Int. Cl. A61K 31/165; A61P 9/12

(52) U.S. Cl. 514/616

(57) ABSTRACT

The present invention relates to methods for the prevention of, delay progression to or treatment of hypertension, comprising administering to a warm-blooded animal a therapeutically effective amount of a renin inhibitor or a pharmaceutically acceptable salt thereof as well as methods of preventing secondary complications linked to cessation of the treatment of hypertension.

The present invention relates to therapeutic methods involving the administration of renin inhibitors, such as aliskiren, or a pharmaceutically acceptable salt thereof. In particular, the present invention provides advantageous methods for treating hypertension comprising in particular aliskiren, preferably, a hemi-fumarate salt thereof.

### INTRODUCTION

In the following the term “aliskiren”, if not defined specifically, is to be understood both as the free base and as a salt thereof especially a pharmaceutically acceptable salt thereof, most preferably a hemi-fumarate thereof.

Renin released from the kidneys cleaves angiotensinogen in the circulation to form the decapeptide angiotensin I. This is in turn cleaved by angiotensin converting enzyme in the lungs, kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume. Inhibitors of the enzymatic activity of renin bring about a reduction in the formation of angiotensin I. As a result a smaller amount of angiotensin II is produced. The reduced concentration of that active peptide hormone is the direct cause of, e.g., the antihypertensive effect of renin inhibitors. Accordingly, renin inhibitors, or salts thereof, may be employed, e.g., as antihypertensives or for treating congestive heart failure and other complications of hypertension such as stroke.

The renin inhibitor, aliskiren, in particular, a hemi-fumarate thereof, is known to be effective as a treatment for reducing blood pressure irrespective of age, sex or race and is also well tolerated. Aliskiren in form of the free base is represented by the following formula

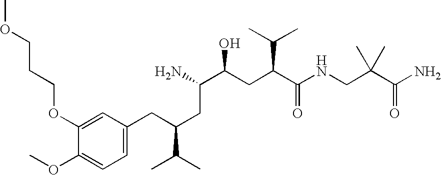

and chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide. As described above, most preferred is the hemi-fumarate salt thereof which is specifically disclosed in EP 678503 A as Example 83.

Although in many cases antihypertensive agents can provide adequate blood pressure control in hypertensive patients, a strict compliance is usually necessary to ensure appropriate blood pressure control. Therapy with antihypertensive agents has to be at regular intervals in order to provide a reliable and lasting blood pressure control. Ideally, antihypertensive agents are administered daily to keep the blood pressure constantly within the desired range. It can be observed, however, that with certain antihypertensive agents a complete 24 h control cannot be achieved or when taken at slightly different times there can be a risk of a lack of continuous blood pressure control. Also, adherence to the prescribed medication regime, known as therapy compliance, is known to be problematic in patients with an essentially a-symptomatic disease like hypertension. Missed doses of anti-hypertensive medication can potentially result in rebound hypertension and to sub-optimal hypertension control, potentially exposing the patient to an increased risk of cardiovascular complications. Mention is made in particular of certain cardiac complications that may arise after missed doses of the antihypertensive agent, in particular in patients already being at a particular risk of such complications such as patients that have suffered previously from myocardial infarction

Given the clinical reality of sub-optimal therapy compliance, it is therefore important to further investigate the persistence of efficacy, withdrawal and rebound effects in order to provide an effective and safe therapy to treat hypertension bearing in mind that the administration of the drug takes place at home in majority of cases so that compliance or lack thereof is an important issue that cannot be controlled sufficiently by the physician.

### SUMMARY OF THE INVENTION

After intense investigations it was found surprisingly that renin inhibitors such as aliskiren, unlike many other antihypertensive agents, have an unexpected high persistence of the blood pressure lowering effect thus providing a safe and efficient therapeutic method for treating hypertension.

The present invention is therefore related to a method for the prevention of, delay progression to or treatment of hypertension, comprising administering to a warm-blooded animal a therapeutically effective amount of a renin inhibitor or a pharmaceutically acceptable salt thereof, whereby an antihypertensive effect is sustained beyond cessation of the administration of the renin inhibitor.

The present invention also relates to a method for the prevention of, delay progression to or treatment of hypertension, comprising administering to a warm-blooded animal a therapeutically effective amount of a renin inhibitor or a pharmaceutically acceptable salt thereof, whereby the blood pressure does not return to baseline levels over a period of at least 5 days after cessation of the administration of the renin inhibitor.

The present invention is further directed to a method for the prevention of, delay progression to or treatment of hypertension, comprising administering to a warm-blooded animal a therapeutically effective amount of a renin inhibitor or a pharmaceutically acceptable salt thereof whereby no rebound hypertension is observed after cessation of the administration of the renin inhibitor.

The present invention is also related to a method of preventing secondary complications linked to cessation of the treatment of hypertension, said method comprising administering to a warm-blooded animal a therapeutically effective amount of a renin inhibitor or a pharmaceutically acceptable salt thereof.

Thus, with the present invention blood pressure is controlled more consistently over time, even if doses are occasionally missed, and there is no evidence of a greater variability of the blood pressure level over time, and therefore poorer outcomes. This is a marked benefit observed with renin inhibitors.

### BRIEF DESCRIPTION OF FIGURES

FIG. 1 describes the mean sitting diastolic blood pressure (mm Hg) during the randomized withdrawal period by treatment group and visit (week)—Long-term study (Randomized withdrawal ITT population) starting from month 11 (visit 10).

FIG. 2 describes the mean sitting systolic blood pressure (mm Hg) during the randomized withdrawal period by treatment group and visit (week)—Long-term study (Randomized withdrawal ITT population) starting from month 11 (visit 10).

FIG. 3 describes the change from baseline in mean sitting diastolic blood pressure (mm Hg) by week and treatment group after 8 weeks of treatment with the indicated amount of aliskiren or placebo.

### DETAILED DESCRIPTION OF THE INVENTION

Listed below are some of the definitions of various additional terms used herein to describe certain aspects of the present invention. However, the definitions used herein are those generally known in the art, e.g., hypertension, and apply to the terms as they are used throughout the specification unless they are otherwise limited in specific instances.

The term “prevention” refers to prophylactic administration to healthy patients to prevent the development of the conditions mentioned herein. Moreover, the term “prevention” means prophylactic administration to patients being in a pre-stage of the conditions to be treated. This is also referred to a primary prevention. In addition the term “prevention” encompasses also “secondary prevention,” which refers to the administration to patients who already have had a condition in order to prevent its recurrence or worsening, or to prevent the complications that may arise from the condition.

The term “delay the onset of”, as used herein, refers to administration to patients being in a pre-stage of the condition to be treated in which patients with a pre-form of the corresponding condition is diagnosed.

The term “treatment” is understood the management and care of a patient for the purpose of combating the disease, condition or disorder.

The term “therapeutically effective amount” refers to an amount of a drug or a therapeutic agent that will elicit the desired biological or medical response of a tissue, system or an animal (including man) that is being sought by a researcher or clinician.

The term “reduced dose that itself is not effective to treat hypertension” refers to an amount of a drug or a therapeutic agent that is too low to elicit the desired biological or medical response of a specific tissue, system or an individual animal (including man) as sought by a researcher or clinician. The reduced dose is a dose specific to the particular subject that is being treated and is specifically a dose that is insufficient for that individual subject to lower the blood pressure to goal blood pressure. Specifically, for the individual animal (including man) the respective chosen dose cannot control hypertension in said animal (including man), in particular, the goal blood pressure of <140 mmHg, systolic pressure and <90 mmHg diastolic pressure. The reduced dose can be any fraction of an effective amount, e.g. in particular for aliskiren, it can be a dose below 75 mg.

The term “synergistic”, as used herein, means that the effect achieved with the methods, combinations and pharmaceutical compositions of the present invention is greater than the sum of the effects that result from individual methods and compositions comprising the active ingredients of this invention separately.

The term “warm-blooded animal or patient” are used interchangeably herein and include, but are not limited to, humans, dogs, cats, horses, pigs, cows, monkeys, rabbits, mice and laboratory animals. The preferred mammals are humans.

The term “pharmaceutically acceptable salt” refers to a non-toxic salt commonly used in the pharmaceutical industry which may be prepared according to methods well-known in the art.

The term “hypertension” refers to a condition where the pressure of blood within the blood vessels is higher than normal as it circulates through the body. When the systolic pressure exceeds 140 mmHg or the diastolic pressure exceeds 90 mmHg for a sustained period of time, damage is done to the body. Populations at increased risk due to other conditions, such as diabetes, are recommended to have even lower levels than cited above. Excessive systolic pressure can rupture blood vessels, and when it occurs within the brain, a stroke results. Hypertension may also cause thickening and narrowing of the blood vessels which ultimately could lead to atherosclerosis. The term “hypertension” as used herein is meant to encompass various types of hypertension, such as those described hereinafter, namely severe hypertension, pulmonary hypertension, malignant hypertension, and isolated systolic hypertension.

The term “severe hypertension” refers to hypertension characterized by a systolic blood pressure of ≧180 mmHg and a diastolic blood pressure of ≧110 mmHg.

The term “pulmonary hypertension” (PH) refers to a blood vessel disorder of the lung in which the pressure in the pulmonary artery rises above normal level of ≦25/10 (especially primary and secondary PH), e.g., because the small vessels that supply blood to the lungs constrict or tighten up. According to the WHO, PH may be divided into five categories: pulmonary arterial hypertension (PAH), a PH occurring in the absence of a known cause is referred to as primary pulmonary hypertension, while secondary PH is caused by a condition selected, e.g., from emphysema; bronchitis; collagen vascular diseases, such as scleroderma, Crest syndrome or systemic lupus erythematosus (SLE); PH associated with disorders of the respiratory system; PH due to chronic thrombotic or embolic disease; PH due to disorders directly affecting the pulmonary blood vessels; and pulmonary venous hypertension (PVH).

The term “malignant hypertension” is usually defined as very high blood pressure with swelling of the optic nerve behind the eye, called papilledema (grade IV Keith-Wagner hypertensive retinopathy). This also includes malignant HTN of childhood.

The term “isolated systolic hypertension” refers to hypertension characterized by a systolic blood pressure of ≧140 mmHg and a diastolic blood pressure of <90 mmHg.

The term “renovascular hypertension” (renal artery stenosis) refers to a condition where the narrowing of the renal artery is significant which leads to an increase of the blood pressure resulting from renin secretion by the kidneys. Biomarkers include renin, PRA and prorenin.

The term “antihypertensive effect” refers to a control of the blood pressure to normal. Preferably, normal blood pressure is characterized by a goal blood pressure of <140 mmHg, preferably <138 mmHg, systolic pressure and <90 mmHg diastolic pressure. In preferred embodiments, the antihypertensive effect refers to a mean sitting diastolic blood pressure of below 89 mm Hg, preferably below 88 mmHg, more preferably 87 mmHg or below. In other preferred embodiments, the antihypertensive effect refers to a mean sitting systolic blood pressure of below 140 mmHg, preferably 139 mmHg, more preferably 138 mmHg or below. Preferably, the antihypertensive effect is sustained for more than 3 days, more preferably more than 10 days, still more preferably more than 21 days, such as 2 to 5 weeks, most preferably, 2, 3, or 4 weeks.

The term “cessation of administration of the renin inhibitor” means withdrawal of the renin inhibitor to treat hypertension. Typically, it refers to complete or intermittent discontinuation of administration of the renin inhibitor, or to administration of a reduced dose of the renin inhibitor that itself is not effective to treat hypertension in the warm-blooded animal. Complete discontinuation means that a therapy with the renin inhibitor is terminated. Intermittent discontinuation means that a therapy with the renin inhibitor is stopped and taken up again after a certain period of time. This can happen when missing a dose or doses or when interrupting the therapy on purpose. The latter may happen for, e.g. certain health and safety reasons. The time period for the intermittent discontinuation may be any suitable time period such as 1 day to several weeks, preferably either a short period of 1 to 6 days, such as 2 to 5 days, or a longer period of 1 to 4 weeks, such as 2 to 3 weeks. Generally speaking, the intermittent cessation can refer to any instance in which the drug is taken less frequently than prescribed. Alternatively, the term “cessation” refers to administration of a reduced dose of the renin inhibitor that itself is not effective to treat hypertension in the warm-blooded animal. The reduced dose is a dose specific to the particular subject that is being treated and is specifically a dose that is insufficient for that individual subject to lower the blood pressure to goal blood pressure, in particular of <140 mmHg, systolic pressure and <90 mmHg diastolic pressure. The reduced dose can be any fraction of an effective amount, e.g. in particular for aliskiren, it can be a dose below 75 mg. Preferably cessation refers to intermittent discontinuation of administration of the renin inhibitor during therapy.

The term “abrupt” in connection with the cessation of administration of the renin inhibitor refers to a cessation without prior adjustment such as gradual reduction of the dose. When a daily treatment regimen is contemplated, an abrupt cessation suitably refers to a cessation from one day to another, whereby one day the therapeutically effective dose is administered and the next day no treatment is provided. Preferably, the antihypertensive effect is sustained after abrupt cessation. Preferably, the blood pressure does not return to baseline over a period of at least 5 days after abrupt cessation. Preferably, no rebound hypertension is observed after abrupt cessation.

The term “baseline level” refers to the blood pressure level of the treated subject prior to the therapy with the renin inhibitor to treat hypertension. The baseline level refers to either or both the systolic and the diastolic blood pressure. Consequently, the baseline level for systolic pressure can be ≧140 mmHg, such as ≧150 mmHg, or ≧160 mmHg, depending on the individual, and the baseline level for the diastolic pressure can be ≧90 mmHg, such as ≧95 mmHg, depending on the individual. Preferably, the blood pressure does not return to baseline levels over a period of at least 5 days, more preferably up to several weeks, such as 2, 3, or 4 weeks.

The term “rebound hypertension” refers to a rise of blood pressure above baseline levels after cessation of administration of the renin inhibitor. Typically rebound hypertension can be encountered within the first days up to within 2 weeks after cessation of an antihypertensive therapy. For the purposes of the studies conducted in connection of the present application, rebound hypertension preferably refers to a rise of >5 mmHg above baseline for DBP and/or >10 mmHg for SBP. Preferably no rebound hypertension is observed over a period of at least 5 days, more preferably up to several weeks, such as 2, 3, or 4 weeks.

The term “secondary complications linked to cessation of the treatment of hypertension” can refer to rebound hypertension. It can also refer to cardiac complications, in particular when patients with a certain risk for developing such complications are treated. Such complications refer in particular to myocardial infarction (MI) including acute MI, and stroke.

Suitable renin inhibitors include compounds having different structural features. For example, mention may be made of compounds which are selected from the group consisting of ditekiren (chemical name: [1S-[1R*,2R*,4R*(1R*,2R*)]]-1-[(1,1-dimethylethoxy)carbonyl]-L-prolyl-L-phenylalanyl-N-[2-hydroxy-5-methyl-1-(2-methylpropyl)-4-[[[2-methyl-1-[[(2-pyridinylmethyl)amino]carbonyl]butyl]amino]carbonyl]hexyl]-N-alfa-methyl-L-histidinamide); terlakiren (chemical name: [R—(R*,S*)]-N-(4-morpholinylcarbonyl)-L-phenylalanyl-N-[1-(cyclohexylmethyl)-2-hydroxy-3-(i-methylethoxy)-3-oxopropyl]-S-methyl-L-cysteineamide); and zankiren (chemical name: [1S-[1R*[R*(R*)],2S*,3R*]]-N-[1-(cyclohexylmethyl)-2,3-dihydroxy-5-methylhexyl]-alfa-[[2-[[(4-methyl-1-piperazinyl)sulfonyl]methyl]-1-oxo-3-phenylpropyl]-amino]4-thiazolepropanamide), preferably, in each case, the hydrochloride salt thereof, SPP630, SPP635 and SPP800 as developed by Speedel.

Preferred renin inhibitor of the present invention include RO 66-1132 and RO 66-1168 of formulae (I) and (II)

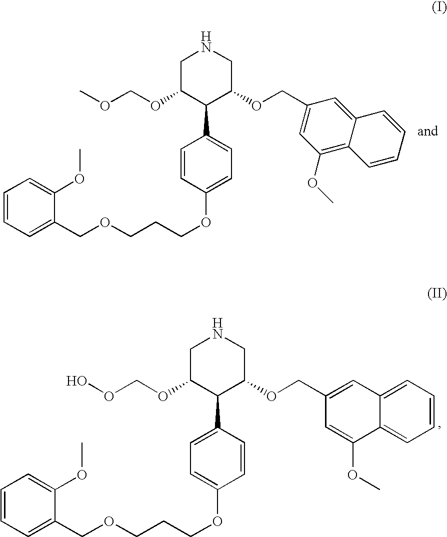

respectively, or a pharmaceutically acceptable salt thereof.

In particular, the present invention relates to a renin inhibitor which is a δ-amino-γ-hydroxy-ω-aryl-alkanoic acid amide derivative of the formula

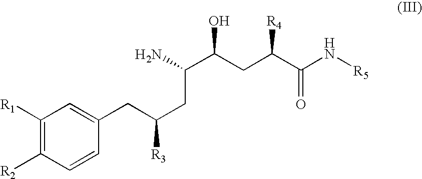

wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

As an alkyl, R₁ may be linear or branched and preferably comprise 1 to 6 C atoms, especially 1 or 4 C atoms. Examples are methyl, ethyl, n- and i-propyl, n-, i- and t-butyl, pentyl and hexyl.

As a halogenalkyl, R₁ may be linear or branched and preferably comprise 1 to 4 C atoms, especially 1 or 2 C atoms. Examples are fluoromethyl, difluoromethyl, trifluoromethyl, chloromethyl, dichloromethyl, trichloromethyl, 2-chloroethyl and 2,2,2-trifluoroethyl.

As an alkoxy, R₁ and R₂ may be linear or branched and preferably comprise 1 to 4 C atoms. Examples are methoxy, ethoxy, n- and i-propyloxy, n-, i- and t-butyloxy, pentyloxy and hexyloxy.

As an alkoxyalkyl, R₁ may be linear or branched. The alkoxy group preferably comprises 1 to 4 and especially 1 or 2 C atoms, and the alkyl group preferably comprises 1 to 4 C atoms. Examples are methoxymethyl, 2-methoxyethyl, 3-methoxypropyl, 4-methoxybutyl, 5-methoxypentyl, 6-methoxyhexyl, ethoxymethyl, 2ethoxyethyl, 3-ethoxypropyl, 4-ethoxybutyl, 5-ethoxypentyl, 6-ethoxyhexyl, propyloxymethyl, butyloxymethyl, 2-propyloxyethyl and 2-butyloxyethyl.

As a C₁₋₆alkoxy-C₁₋₆alkyloxy, R₁ may be linear or branched. The alkoxy group preferably comprises 1 to 4 and especially 1 or 2 C atoms, and the alkyloxy group preferably comprises 1 to 4 C atoms. Examples are methoxymethyloxy, 2-methoxyethyloxy, 3-methoxypropyloxy, 4-methoxybutyloxy, 5-methoxypentyloxy, 6-methoxyhexyloxy, ethoxymethyloxy, 2-ethoxyethyloxy, 3-ethoxypropyloxy, 4-ethoxybutyloxy, 5-ethoxypentyloxy, 6-ethoxyhexyloxy, propyloxymethyloxy, butyloxymethyloxy, 2-propyloxyethyloxy and 2-butyloxyethyloxy.

In a preferred embodiment, R₁ is methoxy- or ethoxy-C₁₋₄alkyloxy, and R₂ is preferably methoxy or ethoxy. Particularly preferred are compounds of formula (III), wherein R₁ is 3-methoxypropyloxy and R₂ is methoxy.

As a branched alkyl, R₃ and R₄ preferably comprise 3 to 6 C atoms. Examples are i-propyl, i- and t-butyl, and branched isomers of pentyl and hexyl. In a preferred embodiment, R₃ and R₄ in compounds of formula (III) are in each case i-propyl.

As a cycloalkyl, R₅ may preferably comprise 3 to 8 ring-carbon atoms, 3 or 5 being especially preferred. Some examples are cyclopropyl, cyclobutyl, cyclopentyl, cyclohexyl and cyclooctyl. The cycloalkyl may optionally be substituted by one or more substituents, such as alkyl, halo, oxo, hydroxy, alkoxy, amino, alkylamino, dialkylamino, thiol, alkylthio, nitro, cyano, heterocyclyl and the like.

As an alkyl, R₅ may be linear or branched in the form of alkyl and preferably comprise 1 to 6 C atoms. Examples of alkyl are listed herein above. Methyl, ethyl, n- and i-propyl, n-, i- and t-butyl are preferred.

As a C₁₋₆hydroxyalkyl, R₅ may be linear or branched and preferably comprise 2 to 6 C atoms. Some examples are 2-hydroxyethyl, 2-hydroxypropyl, 3-hydroxypropyl, 2-, 3- or 4-hydroxybutyl, hydroxypentyl and hydroxyhexyl.

As a C₁₋₆alkoxy-C₁₋₆alkyl, R₅ may be linear or branched. The alkoxy group preferably comprises 1 to 4 C atoms and the alkyl group preferably 2 to 4 C atoms. Some examples are 2-methoxyethyl, 2-methoxypropyl, 3-methoxypropyl, 2-, 3- or 4-methoxybutyl, 2-ethoxyethyl, 2-ethoxypropyl, 3-ethoxypropyl, and 2-, 3- or 4-ethoxybutyl.

As a C₁₋₆alkanoyloxy-C₁₋₆alkyl, R₅ may be linear or branched. The alkanoyloxy group preferably comprises 1 to 4 C atoms and the alkyl group preferably 2 to 4 C atoms. Some examples are formyloxymethyl, formyloxyethyl, acetyloxyethyl, propionyloxyethyl and butyroyloxyethyl.

As a C₁₋₆aminoalkyl, R₅ may be linear or branched and preferably comprise 2 to 4 C atoms. Some examples are 2-aminoethyl, 2- or 3-aminopropyl and 2-, 3- or 4-aminobutyl.

As C₁₋₆alkylamino-C₁₋₆alkyl and C₁₋₆dialkylamino-C₁₋₆alkyl, R₅ may be linear or branched. The alkylamino group preferably comprises C₁₋₄alkyl groups and the alkyl group has preferably 2 to 4 C atoms. Some examples are 2-methylaminoethyl, 2-dimethylaminoethyl, 2-ethylaminoethyl, 2-ethylaminoethyl, 3-methylaminopropyl, 3-dimethylaminopropyl, 4-methylaminobutyl and 4-dimethylaminobutyl.

As a HO(O)C—C₁₋₆alkyl, R₅ may be linear or branched and the alkyl group preferably comprises 2 to 4 C atoms. Some examples are carboxymethyl, carboxyethyl, carboxypropyl and carboxybutyl.

As a C₁₋₆alkyl-O—(O)C—C₁₋₆alkyl, R₅ may be linear or branched, and the alkyl groups preferably comprise independently of one another 1 to 4 C atoms. Some examples are methoxycarbonylmethyl, 2-methoxycarbonylethyl, 3-methoxycarbonylpropyl, 4-methoxycarbonylbutyl, ethoxycarbonylmethyl, 2-ethoxycarbonylethyl, 3-ethoxycarbonylpropyl, and 4-ethoxycarbonylbutyl.

As a H₂N—C(O)—C₁₋₆alkyl, R₅ may be linear or branched, and the alkyl group preferably comprises 2 to 6 C atoms. Some examples are carbamidomethyl, 2-carbamidoethyl, 2-carbamido-2,2-dimethylethyl, 2- or 3-carbamidopropyl, 2-, 3- or 4-carbamidobutyl, 3-carbamido-2-methylpropyl, 3-carbamido-1,2-dimethylpropyl, 3-carbamido-3-ethylpropyl, 3-carbamido-2,2-dimethylpropyl, 2-, 3-, 4- or 5-carbamidopentyl, 4-carbamido-3,3- or -2,2-dimethylbutyl. Preferably, R₅ is 2-carbamido-2,2-dimethylethyl.

Accordingly, preferred are δ-amino-γ-hydroxy-ω-aryl-alkanoic acid amide derivatives of formula (III) having the formula

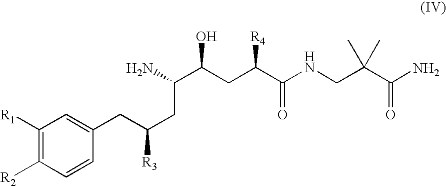

wherein R₁ is 3-methoxypropyloxy; R₂ is methoxy; and R₃ and R₄ are isopropyl; or a pharmaceutically acceptable salt thereof; chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide, also known as aliskiren and as represented by formula (V).

The term “aliskiren”, if not defined specifically, is to be understood both as the free base and as a salt thereof, especially a pharmaceutically acceptable salt thereof, most preferably a hemi-fumarate salt thereof.

The renin inhibitor of formula (V) is preferably in the form of a hemi-fumarate salt.

The structure of the active agents identified by generic or tradenames may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo. The corresponding active ingredients or pharmaceutically acceptable salts thereof may also be used in form of a solvate, such as a hydrate or including other solvents, used for crystallization.

The compounds can be present as pharmaceutically acceptable salts. If these compounds have, for example, at least one basic center, they can form acid addition salts. Corresponding acid addition salts can also be formed having, if desired, an additionally present basic center. Compounds having an acid group (for example COOH) can also form salts with bases.

The compounds may be present in prodrug form. The invention includes prodrugs for the active pharmaceutical species of the invention, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents in particular compounds which are rapidly transformed in vivo to the parent compound, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996), each of which is incorporated herein by reference.

Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. acyloxyalkyl esters, amides |
| Alcohol | Esters, including e.g. sulfates and phosphates as |
| | well as carboxylic acid esters |
| Amine | Amides, carbamates, imines, enamines, |
| Carbonyl (aldehyde, | Imines, oximes, acetals/ketals, enol esters, |
| ketone) | oxazolidines and thiazoxolidines |

Prodrugs also include compounds convertible to the active drug by an oxidative or reductive reaction. As examples may be mentioned:

Oxidative Activation
N- and O-dealkylation
Oxidative deamination
N-oxidation
Epoxidation

Reductive Activation
Azo reduction
Sulfoxide reduction
Disulfide reduction
Bioreductive alkylation
Nitro reduction.

Also to be mentioned as metabolic activations of prodrugs are nucleotide activation, phosphorylation activation and decarboxylation activation. For additional information, see “The Organic Chemistry of Drug Design and Drug Action”, R B Silverman (particularly Chapter 8, pages 497 to 546), incorporated herein by reference.

The use of protecting groups is fully described in ‘Protective Groups in Organic Chemistry’, edited by J W F McOmie, Plenum Press (1973), and ‘Protective Groups in Organic Synthesis’, 2nd edition, T W Greene & P G M Wutz, Wiley-Interscience (1991).

Thus, it will be appreciated by those skilled in the art that, although protected derivatives of compounds of the invention may not possess pharmacological activity as such, they may be administered, for example parenterally or orally, and thereafter metabolised in the body to form compounds of the invention which are pharmacologically active. Such derivatives are therefore examples of “prodrugs”. All prodrugs of the described compounds are included within the scope of the invention.

The pharmaceutical preparations described herein may be for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubulizing or lyophilizing processes. Thus, pharmaceutical preparations for oral use can be obtained by combining the active compound with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical preparation according to the present invention are therapeutically effective dosages, especially those which are commerically available.

Normally, in the case of oral administration, an approximate daily dose of from about 1 mg to about 2 g is to be estimated e.g. for a patient of approximately 75 kg in weight.

The dosage of the active compound can depend on a variety of factors, such as mode of administration, homeothermic species, age and/or individual condition.

The pharmaceutical preparation will usually be supplied in the form of suitable dosage unit form, for example, a capsule or tablet, and comprising an appropriate amount of a combination as disclosed herein.

A solid oral dosage form comprises a capsule or more preferably a tablet or a film-coated tablet.

A solid oral dosage form according to the invention comprises additives or excipients that are suitable for the preparation of the solid oral dosage form according to the present invention. Tabletting aids, commonly used in tablet formulation can be used and reference is made to the extensive literature on the subject, see in particular Fiedler's “Lexicon der Hilfstoffe”, 4th Edition, ECV Aulendorf 1996, which is incorporated herein by reference. These include, but are not limited to, fillers, binders, disintegrants, lubricants, glidants, stabilising agents, fillers or diluents, surfactants, film-formers, softeners, pigments and the like.

In a preferred embodiment the solid oral dosage form according to the present invention comprises as an additive a filler.

In a preferred embodiment the solid oral dosage form according to the present invention comprises as an additive, in addition to a filler, a disintegrant.

In a preferred embodiment the solid oral dosage form according to the present invention comprises as an additive, in addition to a filler and a disintegrant, a lubricant.

In a preferred embodiment the solid oral dosage form according to the present invention comprises as an additive, in addition to a filler, a disintegrant and a lubricant, a glidant.

In a preferred embodiment the solid oral dosage form according to the present invention comprises as an additive, in addition to a filler, a disintegrant, a lubricant and a glidant, a binder.

As fillers one can particularly mention starches, e.g., potato starch, wheat starch, corn starch, hydroxypropyl cellulose, hydroxyethyl cellulose, hydroxypropyl methyl cellulose (HPMC) and, preferably, microcrystalline cellulose, e.g., products available under the registered trade marks AVICEL, FILTRAK, HEWETEN or PHARMACEL.

As binders for wet granulation, one can particularly mention polyvinylpyrrolidones (PVP), e.g., PVP K 30, HPMC, e.g., viscosity grades 3 or 6 cps, and polyethylene glycols (PEG), e.g., PEG 4000. A most preferred binder is PVP K 30.

As disintegrants one can particularly mention carboxymethylcellulose calcium (CMC-Ca), carboxymethylcellulose sodium (CMC-Na), crosslinked PVP (e.g. CROSPOVIDONE, POLYPLASDONE or KOLLIDON XL), alginic acid, sodium alginate and guar gum, most preferably crosslinked PVP (CROSPOVIDONE), crosslinked CMC (Ac-Di-Sol), carboxymethylstarch-Na (PIRIMOJEL and EXPLOTAB). A most preferred disintegrant is CROSPOVIDONE.

As glidants one can mention in particular colloidal silica, such as colloidal silicon dioxide, e.g., AEROSIL, magnesium (Mg) trisilicate, powdered cellulose, starch, talc and tribasic calcium phosphate or combinations of these with fillers or binders, e.g., silicified microcrystalline cellulose (PROSOLV). A most preferred glidant is colloidal silicon dioxide (e.g. AEROSIL 200).

As fillers or diluents one can mention confectioner's sugar, compressible sugar, dextrates, dextrin, dextrose, lactose, mannitol, microcrystalline cellulose, in particular, having a density of about 0.45 g/cm³, e.g., AVICEL, powdered cellulose, sorbitol, sucrose and talc. A most preferred filler is microcrystalline cellulose.

As lubricants one can mention in particular Mg stearate, aluminum (Al) or Ca stearate, PEG 4000 to 8000 and talc, hydrogenated castor oil, stearic acid and salts thereof, glycerol esters, Na-stearylfumarate, hydrogenated cotton seed oil and others. A most preferred lubricant is Mg stearate.

Additives to be used as filmcoating materials comprise polymers such as HPMC, PEG, PVP, polyvinylpyrrolidone-vinyl acetate copolymer (PVP-VA), polyvinyl alcohol (PVA), and sugar as film formers. A most preferred coating material is HPMC, especially HPMC 3 cps (preferred amount 5-6 mg/cm²), and mixtures thereof with further additives, e.g., those available under the registered trade mark OPADRY. Further additives comprise pigments, dies, lakes, most preferred TiO₂ and iron oxides, anti-tacking agents like talk and softeners like PEG 3350, 4000, 6000, 8000 or others. Most preferred additives are talk and PEG 4000.

The doses of renin inhibitor such as one of formula (V) to be administered to warm-blooded animals, for example human beings, of, for example, approximately 70 kg body weight, especially the doses effective in the inhibition of the enzyme renin, e.g. in lowering blood pressure may be from approximately 3 mg to approximately 3 g, particularly from approximately 10 mg to approximately 1 g, for example approximately from 20 mg to 600 mg (e.g. 150 mg to 300 mg), per person per day. Single doses comprise, for example, 75, 100, 150, 200, 250, 300 or 600 mg per adult patient. Usually, children receive about half of the adult dose or they can receive the same dose as adults. The dose necessary for each individual can be monitored and adjusted to an optimum level. The usual recommended starting dose of a renin inhibitor of formula (V) is usually 150 mg once daily. In some patients whose blood pressure is not adequately controlled, the daily dose may be increased to 300 mg. The renin inhibitor of formula (V) may be used over a dosage range of 150 mg to 300 mg administered once daily.

Ultimately, the exact dose of the active agent and the particular formulation to be administered depend on a number of factors, e.g., the condition to be treated, the desired duration of the treatment and the rate of release of the active agent. For example, the amount of the active agent required and the release rate thereof may be determined on the basis of known in vitro or in vivo techniques, determining how long a particular active agent concentration in the blood plasma remains at an acceptable level for a therapeutic effect.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way.

### EXAMPLE 1

Composition of aliskiren 150 mg (free base) uncoated tablets in mg/unit.

| | Roller | | | |
|---|---|---|---|---|
| | compacted | Dosage | Dosage | Dosage |
| Component | tablet | form 1 | form 2 | form 3 |
| Aliskiren hemi-fumarate | 165.750 | 165.750 | 165.750 | 165.750 |
| Microcrystalline cellulose | 220.650 | 84.750 | 72.250 | 107.250 |
| Polyvinylpyrrolidon K 30 | — | — | 12.000 | 12.000 |
| Crospovidone | 84.000 | 45.000 | 44.000 | 48.200 |
| Aerosil 200 | 4.800 | 1.500 | 1.500 | 1.800 |
| Magnesium stearate | 4.800 | 3.000 | 4.500 | 5.000 |
| Total weight | 480.000 | 300.000 | 300.000 | 340.000 |

Composition of aliskiren 150 mg (free base) uncoated tablets in % by weight.

| | Roller | | | |
|---|---|---|---|---|
| | compacted | Dosage | Dosage | Dosage |
| Component | tablet | form 1 | form 2 | form 3 |
| Aliskiren hemi-fumarate | 34.53 | 55.25 | 55.25 | 48.75 |
| Microcrystalline cellulose | 45.97 | 28.25 | 24.08 | 31.545 |
| Polyvinylpyrrolidon K 30 | — | — | 4 | 3.53 |
| Crospovidone | 17.5 | 15 | 14.67 | 14.175 |
| Aerosil 200 | 1 | 0.5 | 0.5 | 0.53 |
| Magnesium stearate | 1 | 1 | 1.5 | 1.47 |
| Total % | 100.00 | 100.00 | 100.00 | 100.00 |

Composition of aliskiren 150 mg (free base) uncoated tablets in mg/unit (divided into inner/outer phase).

| | | Roller | | | |
|---|---|---|---|---|---|
| | | compacted | Dosage | Dosage | Dosage |
| | | tablet | form 1 | form 2 | form 3 |
| Component | | | | | |
| Inner | Aliskiren | 165.75 | 165.75 | 165.75 | 165.75 |
| Phase | hemi-fumarate | | | | |
| | Microcrystalline | 220.65 | 84.75 | 72.25 | 90.25 |
| | cellulose | | | | |
| | Polyvinylpyrrolidon | — | — | 12.00 | 12.00 |
| | K 30 | | | | |
| | Crospovidone | 36.00 | — | — | 14.20 |
| | Aerosil 200 | — | — | — | — |
| | Magnesium stearate | 2.40 | — | — | — |
| Outer | Crospovidone | 48.00 | 45.00 | 44.00 | 34.00 |
| phase | Microcrystalline | — | — | — | 17.00 |
| | cellulose | | | | |
| | Aerosil 200 | 4.80 | 1.50 | 1.50 | 1.80 |
| | Magnesium stearate | 2.40 | 3.00 | 4.50 | 5.00 |
| | Total weight | 480.00 | 300.00 | 300.00 | 340.00 |

Composition of aliskiren 150 mg (free base) uncoated tablets in % by weight (divided into inner/outer phase).

| | | Roller | | | |
|---|---|---|---|---|---|
| | | compacted | Dosage | Dosage | Dosage |
| | | tablet | form 1 | form 2 | form 3 |
| Component | | | | | |
| Inner | Aliskiren | 34.53 | 55.25 | 55.25 | 48.75 |
| Phase | hemi-fumarate | | | | |
| | Microcrystalline | 45.97 | 28.25 | 24.08 | 26.545 |
| | cellulose | | | | |
| | Polyvinylpyrrolidon | — | — | 4 | 3.530 |
| | K 30 | | | | |
| | Crospovidone | 7.5 | — | — | 4.175 |
| | Aerosil 200 | — | — | — | — |
| | Magnesium stearate | 0.5 | — | — | — |
| Outer | Crospovidone | 10 | 15 | 14.67 | 10 |
| phase | Microcrystalline | — | — | — | 5 |
| | cellulose | | | | |
| | Aerosil 200 | 1 | 0.5 | 0.5 | 0.53 |
| | Magnesium stearate | 0.5 | 1 | 1.5 | 1.47 |
| | Total % | 100.00 | 100.00 | 100.00 | 100.00 |

### EXAMPLE 2

Composition of aliskiren (dosage form 3) film-coated tablets in mg/unit.

| | Dosage form 3/Strength | | |
|---|---|---|---|
| | 75 mg | 150 mg | 300 mg |
| Component | (free base) | (free base) | (free base) |
| Aliskiren hemi-fumarate | 82.875 | 165.750 | 331.500 |
| Microcrystalline cellulose | 53.625 | 107.250 | 214.500 |
| Polyvinylpyrrolidon K 30 | 6.000 | 12.000 | 24.000 |
| Crospovidone | 24.100 | 48.200 | 96.400 |
| Aerosil 200 | 0.900 | 1.800 | 3.600 |
| Magnesium stearate | 2.500 | 5.000 | 10.000 |
| Total tablet weight | 170.000 | 340.000 | 680.000 |
| Opadry premix white | 9.946 | 16.711 | 23.9616 |
| Opadry premix red | 0.024 | 0.238 | 1.8382 |
| Opadry premix black | 0.030 | 0.051 | 0.2002 |
| Total fim-coated tablet | 180.000 | 357.000 | 706.000 |
| weight | | | |

### EXAMPLE 3

### Clinical Studies

Persistence of effect was examined after a long-term study in which patients were treated for approximately 1 year. Patients were randomly assigned to aliskiren at either 150 mg or 300 mg a day. Aliskiren was increased to 300 mg in those patients who had not responded to 150 mg. Starting at the end of treatment month 2 and 3, investigators adjusted individual therapy in order to achieve a goal BP of <140/90 mm Hg. Subsequent upward dose titration required that BP exceed 140/90 for two consecutive visits. No downward titration of aliskiren was permitted.

At month 11 (visit 10), patients who had been maintained on aliskiren monotherapy entered a placebo-controlled randomized withdrawal study to confirm continued efficacy. Eligible patients were stratified based on dose and randomized in a 1:1 ratio to either continue their current treatment of aliskiren monotherapy (aliskiren 150 mg or aliskiren 300 mg) or be switched to placebo in a double-blind fashion.

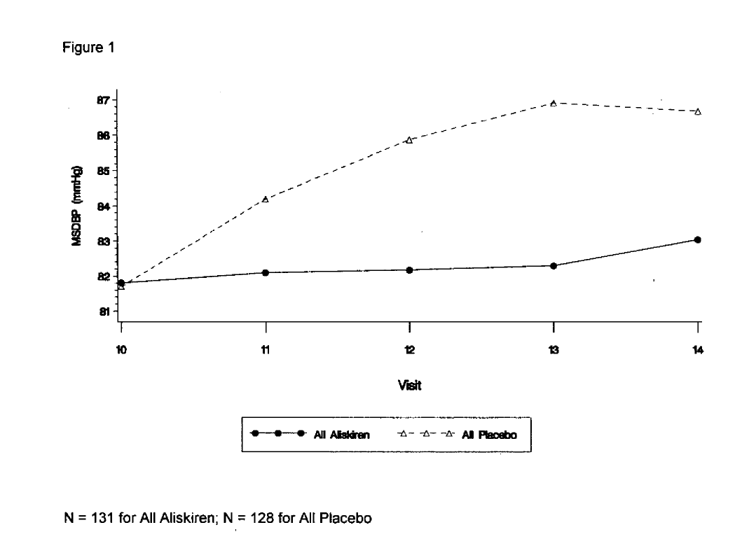

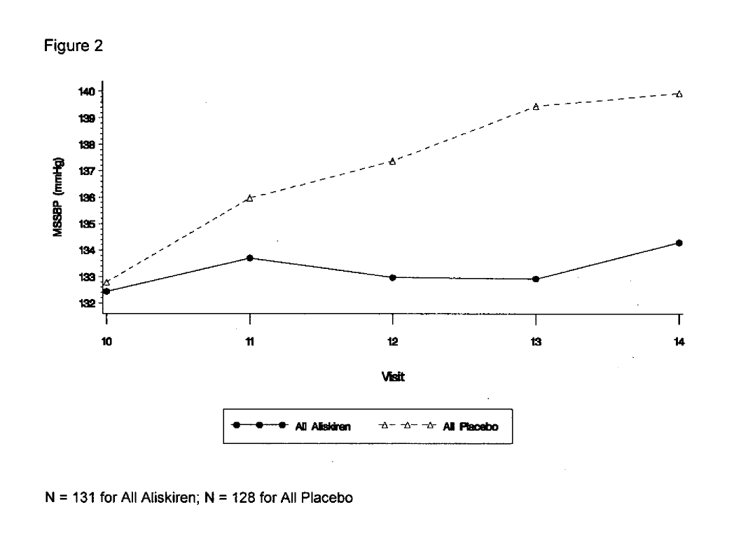

The results on persistence of effect after withdrawal and lack of rebound hypertension are shown in Table 1 and in FIGS. 1 and 2. Although the antihypertensive effect decreased somewhat in the placebo group compared to the aliskiren group, a persistence of a blood pressure lowering effect was observed for several weeks after withdrawal commencing at month 11 (visit 10 as shown in FIGS. 1 and 2).

The potential for a rebound effect on BP with abrupt treatment withdrawal was evaluated.

Rebound was defined as a rise of >5 mm Hg for diastolic and >10 mm for systolic was used and the results are presented separately. There was no sign of rebound on withdrawal of aliskiren treatment.

TABLE 1
Mean changes from Month 11 (Visit 10) in msDBP and msSBP (mm Hg) at randomized withdrawal
visit by treatment group in long-term study (Randomized withdrawal ITT population)

| | | All Aliskiren | | | All Placebo | | |
|---|---|---|---|---|---|---|---|
| | | N = 131 | | | N = 128 | | |
| Randomized withdrawal | | | Change (SD) | | | Change (SD) | |
| Visit | Month | N* | msDBP | msSBP | N* | msDBP | msSBP |
| 11 | Month 11 + 7 Days | 131 | 0.3 (6.8) | 1.3 (10.3) | 128 | 2.5 (8.7) | 3.2 (11.3) |
| 12 | Month 11 + 14 Days | 129 | 0.3 (6.8) | 0.3 (10.4) | 126 | 4.2 (10.1) | 4.7 (13.8) |
| 13 | Month 11 + 21 Days | 127 | 0.4 (6.7) | 0.5 (10.6) | 124 | 5.1 (9.9) | 6.7 (13.3) |
| 14 | Month 11 + 28 Days | 125 | 1.1 (6.8) | 1.8 (10.1) | 123 | 4.8 (10.0) | 7.0 (13.3) |
| End point** | | 131 | 1.2 (6.9) | 1.7 (10.1) | 128 | 5.0 (10.1) | 7.4 (13.6) |

*N is the number of patients with values obtained at both Month 11 (Visit 10) and post-Month 11 (Visit 10) visit.
**Endpoint is Month 11 + 28 days, or last visit carried forward.
Note:
A decrease in the mean change indicates improvement.

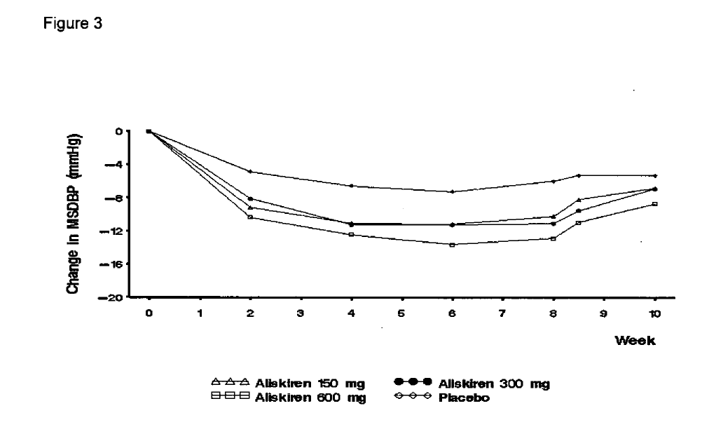

In another study, the potential for rebound hypertension following abrupt withdrawal of aliskiren treatment was evaluated in those patients who completed eight weeks of treatment compared to patients that were given a placebo only. The tested doses were 150, 300 and 600 mg of aliskiren. As shown in FIG. 3, BPs increased in all active treatment groups after the withdrawal of study drug, but values still remained lower than those in the placebo group throughout the drug withdrawal period. At 4 days and 2 weeks after drug withdrawal (last two entries in FIG. 3), more patients in the aliskiren 150 mg, 300 mg, and 600 mg groups had msDBP and msSBP values that remained below baseline compared with the placebo group. This demonstrates that a good persistence of the antihypertensive effect following withdrawal of aliskiren treatment is observed at least for the period of study of two weeks and that there are no signs of rebound hypertension.

1. A method for the prevention of, delay progression to or treatment of hypertension, comprising administering to a warm-blooded animal a therapeutically effective amount of a renin inhibitor or a pharmaceutically acceptable salt thereof, whereby an antihypertensive effect is sustained beyond cessation of the administration of the renin inhibitor.

2. The method of claim 1, wherein the renin inhibitor is a compound of formula (I) 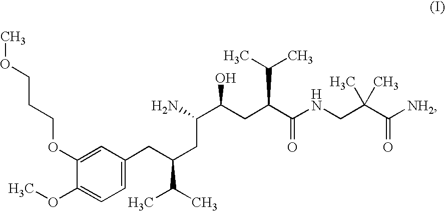
or a pharmaceutically acceptable salt thereof.

3. The method of claim 1 whereby the antihypertensive effect is sustained for more than 3 days.

4. The method of claim 1 whereby the antihypertensive effect is sustained for more than 10 days.

5. The method of claim 1 whereby the antihypertensive effect is sustained for more than 21 days.

6. The method of claim 1 whereby the antihypertensive effect is sustained for 2 to 5 weeks.

7. The method of claim 1 whereby the antihypertensive effect is sustained for 4 weeks.

8. The method of claim 1 whereby the antihypertensive effect is sustained beyond abrupt cessation.

9. The method of claim 1 whereby the antihypertensive effect is sustained beyond cessation from one day to another.

10. The method of claim 1 whereby cessation refers to complete or intermittent discontinuation of administration of the renin inhibitor, or to administration of a reduced dose of the renin inhibitor that itself is not effective to treat hypertension in the warm-blooded animal.

11. The method of claim 1 whereby cessation refers to intermittent discontinuation of administration of the renin inhibitor during therapy.

12. The method of claim 1 whereby the antihypertensive effect refers to a mean sitting diastolic blood pressure of below 90 mm Hg.

13. The method of claim 1 whereby the antihypertensive effect refers to a mean sitting diastolic blood pressure of below 88 mm Hg.

14. The method of claim 1 whereby the antihypertensive effect refers to a mean sitting systolic blood pressure of below 140 mm Hg.

15. The method of claim 1 whereby the antihypertensive effect refers to a mean sitting systolic blood pressure of below 138 mm Hg.

16. A method for the prevention of, delay progression to or treatment of hypertension, comprising administering to a warm-blooded animal a therapeutically effective amount of a renin inhibitor or a pharmaceutically acceptable salt thereof, whereby the blood pressure does not return to baseline levels over a period of at least 5 days after cessation of the administration of the renin inhibitor.

17. The method of claim 16, wherein the renin inhibitor is a compound of formula (I) 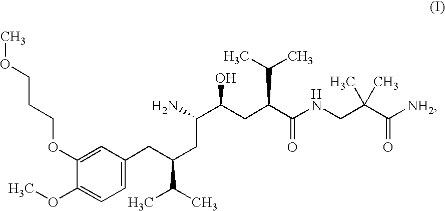
or a pharmaceutically acceptable salt thereof.

18. The method of claim 16 whereby the blood pressure did not return to baseline levels over a period of up to 4 weeks.

19. The method of claim 16 whereby the blood pressure does not return to baseline levels over a period of at least 5 days after abrupt cessation.

20. The method of claim 16 whereby the blood pressure does not return to baseline levels over a period of at least 5 days after cessation from one day to another.

21. The method of claim 16 whereby cessation refers to complete or intermittent discontinuation of administration of the renin inhibitor, or to administration of a reduced dose of the renin inhibitor that itself is not effective to treat hypertension in the warm-blooded animal.

22. The method of claim 16 whereby cessation refers to intermittent discontinuation of administration of the renin inhibitor during therapy.

23. A method for the prevention of, delay progression to or treatment of hypertension, comprising administering to a warm-blooded animal a therapeutically effective amount of a renin inhibitor or a pharmaceutically acceptable salt thereof, whereby no rebound hypertension is observed after cessation of the administration of the renin inhibitor.

24. The method of claim 23, wherein the renin inhibitor is a compound of formula (I) 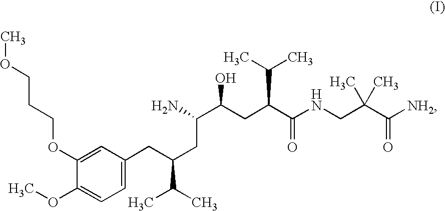
or a pharmaceutically acceptable salt thereof.

25. The method of claim 23 whereby no rebound hypertension is observed over a period of up to 4 weeks.

26. The method of claim 23 whereby no rebound hypertension is observed after abrupt cessation.

27. The method of claim 23 whereby no rebound hypertension is observed after cessation from one day to another.

28. The method of claim 23 whereby cessation refers to complete or intermittent discontinuation of administration of the renin inhibitor, or to administration of a reduced dose of the renin inhibitor that itself is not effective to treat hypertension in the warm-blooded animal.

29. The method of claim 23 whereby cessation refers to intermittent discontinuation of administration of the renin inhibitor during therapy.

30. The method of claim 23 whereby rebound hypertension refers to a diastolic blood pressure or a systolic blood pressure greater than baseline at any time during withdrawal.

31. The method of claim 23 whereby rebound hypertension refers to a diastolic blood pressure >5 mm Hg above baseline.

32. The method of claim 23 whereby rebound hypertension refers to a systolic blood pressure >10 mm Hg above baseline.

33. A method of preventing secondary complications linked to cessation of the treatment of hypertension, said method comprising administering to a warm-blooded animal a therapeutically effective amount of a renin inhibitor or a pharmaceutically acceptable salt thereof.

34. The method of claim 33, wherein the renin inhibitor is a compound of formula (I) 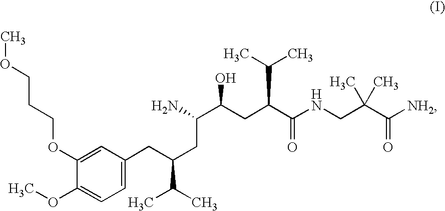
or a pharmaceutically acceptable salt thereof.

35. The method of claim 33 whereby the secondary complication is rebound hypertension.

36. The method of claim 33 whereby the secondary complication is a cardiac complication such as one selected from the group consisting of myocardial infarction and stroke.

37. The method of claim 33 whereby the cessation refers to abrupt cessation.

38. The method of claim 33 whereby cessation takes place from one day to another.

39. The method of claim 33 whereby cessation refers to complete or intermittent discontinuation of administration of the renin inhibitor, or to administration of a reduced dose of the renin inhibitor that itself is not effective to treat hypertension in the warm-blooded animal.

40. The method of claim 33 whereby cessation refers to intermittent discontinuation of administration of the renin inhibitor during therapy.
